(19) United States
(12) Patent Application Publication
(10) Pub. No.: US 2015/0004239 A1
(43) Pub. Date: Jan. 1, 2015

(54) PHARMACEUTICAL COMPOSITIONS AND METHODS FOR THEIR PREPARATION

(71) Applicant: Gilead Sciences, Inc., Foster City, CA (US)

(72) Inventors: Aaron J. Cullen, Mililani, HI (US); Richard Hung Chiu Yu, San Francisco, CA (US)

(21) Appl. No.: 14/371,716

(22) PCT Filed: Jan. 11, 2013
(86) PCT No.: PCT/US2013/021267

§ 371 Date: Jul. 10, 2014

Related U.S. Application Data
(60) Provisional application No. 61/585,920, filed on Jan. 12, 2012.

(51) Int. Cl. A61K 9/50; A61K 31/47; A61K 31/4418; A61K 31/513; A61K 47/22; A61K 31/635; A61K 31/675

(52) U.S. Cl. CPC A61K 9/5015; A61K 31/635; A61K 31/47; A61K 31/675; A61K 31/513; A61K 47/22; A61K 31/4418
USPC 424/490; 514/158; 514/312; 514/81; 514/274; 514/357

(57) ABSTRACT

The invention provides solid particles comprising: a) a solid core that comprises an active pharmaceutical agent and b) a coating of Compound 2: (2) or a pharmaceutically acceptable salt of thereof on the core, as well as compositions comprising such particles, and methods for treating diseases (e.g. HIV infection) with such particles.

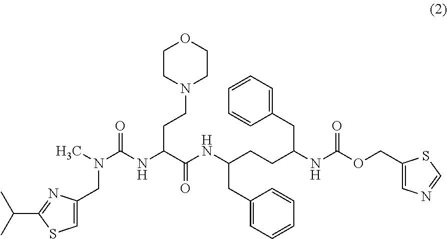 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]

### BACKGROUND OF THE INVENTION

International patent application publication number WO 2008/010921 describes compounds and pharmaceutical compositions that improve the pharmacokinetics of a co-administered drug by inhibiting cytochrome P450 monooxygenase. One such inhibitor is Compound 2.

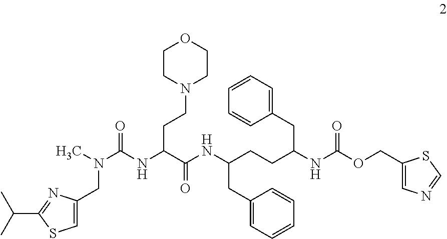 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]

Unfortunately, the solid state properties of Compound 2 make it difficult to handle and process on a large scale. For example, its low glass transition temperature, hygroscopicity, and lack of crystallinity, as well as its non free-flowing nature make it particularly difficult to process and to formulate (e.g. as a tablet).

International patent application publication number WO 2009/135,179 discusses the difficulties associated with processing of Compound 2 and describes combining Compound 2 with certain solid carrier particles to improve the physical properties of the resulting solid material. Although the resulting free-flowing powder has high loading values for Compound 2, acceptable physical and chemical stability, rapid drug release properties, and excellent compressibility, the inert carrier particles contribute to the overall weight and volume of the solid so that significantly more material is required in a formulation to achieve a given dose of Compound 2. Accordingly, there is a need for other solid forms of Compound 2 that have the beneficial properties of the solids described in WO 2009/135,179, but lack the inert carrier particles that contribute to the weight and the volume of the solid. There is also a need for improved formulations that include Compound 2 and one or more pharmaceutical agents that are metabolized by cytochrome P450 monooxygenase.

### SUMMARY OF THE INVENTION

It has now been determined that when Compound 2 or a pharmaceutically acceptable salt thereof is combined with (e.g. coated on) solid particles comprising an active pharmaceutical agent, the resulting combination possesses beneficial physical properties. For example, the resulting combination is typically a free-flowing powder, with high loading values for Compound 2, acceptable physical and chemical stability, rapid drug release properties, and excellent compressibility. Thus, the resulting combination can readily be processed into solid dosage forms (e.g. tablets), which possess acceptable drug release properties, low tablet friability, good chemical and physical stability, and a low amount of residual solvents. The compositions represent a significant advance that facilitates the commercial development of formulations comprising Compound 2 and one or more active pharmaceutical agents.

Accordingly, in one embodiment there is a particle comprising: a) a solid core that comprises an active pharmaceutical agent and b) a coating of Compound 2:

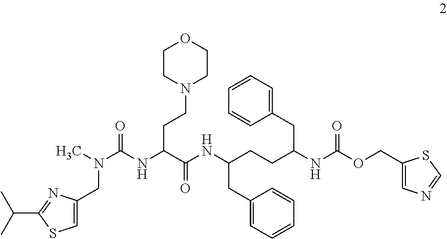 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]

or a pharmaceutically acceptable salt thereof on the core.

In another embodiment, there is a composition comprising a plurality of solid particles.

In another embodiment there is a pharmaceutical composition comprising a plurality of solid particles and a pharmaceutically acceptable excipient.

In another embodiment there is a tablet comprising a plurality of solid particles.

In another embodiment there is a method for treating an HIV infection comprising administering to a patient in need thereof a therapeutically effective amount of a composition wherein the therapeutically active agent is an anti-HIV agent.

In another embodiment there is a method for treating an HIV infection comprising administering to a patient in need thereof a therapeutically effective amount of a composition, wherein the therapeutically active agent is an anti-HIV agent, in combination with a therapeutically effective amount of one or more other therapeutic agents selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, and CCR5 inhibitors.

In another embodiment there is a composition for use in medical therapy.

In another embodiment there is a use of a composition of the invention wherein the therapeutically active agent is an anti-HIV agent for the prophylactic or therapeutic treatment of an HIV infection.

In another embodiment there is a composition wherein the therapeutically active agent is an anti-HIV agent for use in the preparation of a medicament for treating HIV infection in a mammal.

In another embodiment there is a method comprising: a) combining 6-(3-chloro-2-fluorobenzyl)-1-[(2S)-1-hydroxy-3-methylbutan-2-yl]-7-methoxy-4-oxo-1,4-dihydroquinoline-3-carboxylic acid or a salt thereof, and Compound 2:

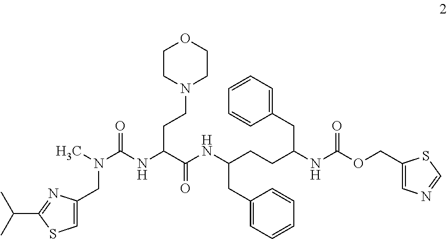 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]

in a suitable solvent to provide a mixture; and b) maintaining the mixture under conditions suitable to provide one or more solid particles comprising 6-(3-chloro-2-fluorobenzyl)-1-[(2S)-1-hydroxy-3-methylbutan-2-yl]-7-methoxy-4-oxo-1,4-dihydroquinoline-3-carboxylic acid or a salt thereof, and having a coating of Compound 2 or a pharmaceutically acceptable salt thereof.

In another embodiment there is a method for preparing a pharmaceutical composition comprising combining a plurality of solid particles and a pharmaceutically acceptable excipient to provide the pharmaceutical composition.

In another embodiment there is a method for preparing a pharmaceutical composition comprising combining tenofovir disoproxil fumarate, emtricitabine and a plurality of solid particles wherein the active pharmaceutical agent is elvitegravir, to provide the pharmaceutical composition.

In another embodiment there is a method for preparing a pharmaceutical composition comprising combining GS-7340, emtricitabine, and a plurality of solid particles wherein the active pharmaceutical agent is elvitegravir, to provide the pharmaceutical composition.

In another embodiment there is a method comprising: a) combining an active pharmaceutical agent and Compound 2:

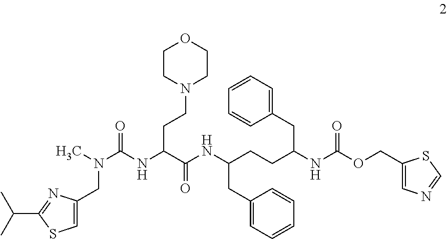 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]

in a suitable solvent to provide a mixture; and b) maintaining the mixture under conditions suitable to provide one or more solid particles comprising a solid core that comprises the active pharmaceutical agent and a coating of Compound 2.

In another embodiment there is a pharmaceutical composition comprising:

a core comprising 6-(3-chloro-2-fluorobenzyl)-1-[(2S)-1-hydroxy-3-methylbutan-2-yl]-7-methoxy-4-oxo-1,4-dihydroquinoline-3-carboxylic acid or a salt thereof; and

the core having a coating including Compound 2:

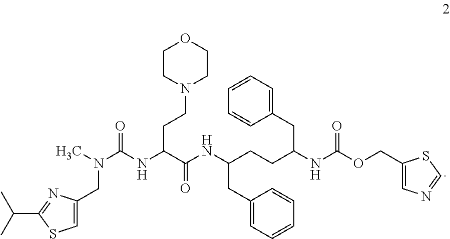 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]

In another embodiment there is a material prepared by a method described herein.

### BRIEF DESCRIPTION OF THE FIGURES

Exemplary implementations of the present invention will now be described with reference to the accompanying drawings, wherein:

FIG. 1 illustrates results for thermal analysis by differential scanning calorimetry as measured in Example 2.

FIG. 2 illustrates results for thermal analysis by thermal gravimetric analysis as measured in Example 2.

FIG. 3 illustrates results for hygroscopicity measurements from Example 2.

FIG. 4 illustrates a morphology of the product of Example 1 observed using SEM (500×).

FIG. 5 illustrates results from X-Ray powder diffraction measurements from Example 2; y-axis (intensity (counts)) and x-axis (2Theta); top line represents the XRPD pattern for the γ form of Compound 5; and the bottom line represents the XRPD pattern for the γ form of Compound 5 containing amorphous material (consistent with the amorphous form of compound 2a).

FIG. 6 illustrates results from X-Ray powder diffraction measurements from Example 2; y-axis (intensity (counts)) and x-axis (2Theta)

FIG. 7 is a cross-sectional view of a core and coating in accordance with one or more embodiments.

FIG. 8 is a cross-sectional view of a core and coating in accordance with one or more embodiments.

FIG. 9 is a flow-diagram that illustrates the preparation of particles of the invention using high sheer wet milling.

In the Figures, the abbreviation COBI is used for Compound 2 and the abbreviation EVG is used for elvitegravir.

### DETAILED DESCRIPTION OF THE INVENTION

It will be appreciated by those skilled in the art that Compound 2 may exist in and be isolated in optically active and racemic forms. Some compounds may exhibit polymorphism. It is to be understood that any racemic, optically-active, polymorphic, or stereoisomeric form, or mixtures thereof, of Compound 2 are included which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

The compositions may include one or more pharmaceutically acceptable excipients. Excipients include but are not limited to substances that can serve as a vehicle or medium for a composition of the invention (e.g. a diluent, carrier, binder, filler, disintegrating agent, lubricant, sweetening agent or flavoring agent). They may be enclosed in hard or soft shell gelatin capsules, may be compressed into tablets, or may be incorporated directly with the food of the patient's diet. For oral therapeutic administration, the composition may be combined with one or more excipients and used in the form of ingestible tablets, buccal tablets, troches, capsules, elixirs, suspensions, syrups, wafers, and the like. Such compositions and preparations will typically contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may contain the following: binders such as hydroxypropyl cellulose, povidone, or hydroxypropyl methylcellulose; fillers, such as microcrystalline cellulose, pregelatinized starch, starch, mannitol, or lactose monohydrate; a disintegrating agent such as croscarmellose sodium, cross-linked povidone, or sodium starch glycolate; a lubricant such as magnesium stearate, stearic acid, or other metallic stearates; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, polymers, wax, shellac or sugar and the like. Of course, any material used in preparing any unit dosage form will typically be pharmaceutically acceptable and substantially non-toxic in the amounts employed. In addition, the compositions of the invention may be incorporated into sustained-release preparations and devices.

The compositions of the invention can also be administered topically, e.g., transdermally, buccally, or sublingually. Accordingly, the invention also provides pharmaceutical compositions that are formulated for such routes of topical administration. Useful dosages can be determined by comparing in vitro activity, and in vivo activity in animal models. Methods for the extrapolation of effective dosages in mice, and other animals, to humans are known to the art.

The amount of a composition of the invention required for use in treatment will vary with the route of administration, the nature of the condition being treated and the age and condition of the patient and will be ultimately at the discretion of the attendant physician or clinician.

The particles of the invention can conveniently be formulated in unit dosage form; for example, containing about 5 to 500 mg, about 5 to 250 mg, or about 10 to 100 mg of the active therapeutic agent. In one embodiment, the invention provides a composition comprising about 5, about 25, or about 100 mg of the active therapeutic agent formulated in a unit dosage, and one or more pharmaceutically acceptable excipients.

### Active Therapeutic Agents

It has been determined that Compound 2 can be coated onto a solid core that comprises one or more active pharmaceutical agents to provide a particle that has beneficial physical properties. The core can also consist or consist essentially of an active pharmaceutical agent or one or more active pharmaceutical agents. The core can be uniform or variable in thickness. For example, the resulting particles typically provide a free-flowing powder, with acceptable physical and chemical stability, rapid drug release properties, and excellent compressibility. Thus, the resulting particles can readily be processed into solid dosage forms (e.g. tablets), which possess acceptable drug release properties, low tablet friability, good chemical and physical stability, and a low amount of residual solvents. Accordingly, the particles represent a significant advance that facilitates the commercial development of formulations comprising Compound 2 and one or more active pharmaceutical agents.

Compound 2 inhibits cytochrome P450 monooxygenase activity. Accordingly, Compound 2 can be administered in combination with one or more active pharmaceutical agents that are metabolized by cytochrome P450 monooxygenase. Any active pharmaceutical agent can be incorporated into the cores of the particles provided the resulting particles have beneficial physical or therapeutic properties. For example, the active pharmaceutical agent in the core can be an agent that is metabolized by cytochrome P450 monooxygenase. Alternatively, the active pharmaceutical agent can be an agent that is typically administered as part of a combination therapy with another agent that is metabolized by cytochrome P450 monooxygenase, although the pharmaceutical agent itself is not significantly metabolized by cytochrome P450 monooxygenase.

In one embodiment the active pharmaceutical agent is an agent that is metabolized by cytochrome P450 monooxygenase when administered to an animal.

In one embodiment the active pharmaceutical agent is an agent that is not metabolized by cytochrome P450 monooxygenase when administered to an animal.

In one embodiment the active pharmaceutical agent is an anti-viral agent.

In one embodiment the active pharmaceutical agent is selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp 120 inhibitors, CCR5 inhibitors, capsid polymerization inhibitors, interferons, ribavirin analogs, NS3 protease inhibitors, alpha-glucosidase 1 inhibitors, hepatoprotectants, non-nucleoside inhibitors of HCV, and other drugs for treating HCV, and combinations thereof.

In one embodiment the active pharmaceutical agent is an anti-HIV agent.

In one embodiment the active pharmaceutical agent is a protease inhibitor.

In one embodiment the active pharmaceutical agent is an integrase inhibitor.

In one embodiment the active pharmaceutical agent is:

1) amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, GW640385X, DG17, GS-8374, PPL-100, DG35, or AG 1859,

2) a HIV non-nucleoside inhibitor of reverse transcriptase, e.g., capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirene), efavirenz, BILR 355 BS, VRX 840773, UK-453061, or RDEA806,

3) a HIV nucleoside inhibitor of reverse transcriptase, e.g., zidovudine, emtricitabine, didanosine, stavudine, zalcitabine, lamivudine, abacavir, amdoxovir, elvucitabine, alovudine, MIV-210, racivir (±-FTC), D-d4FC, emtricitabine, phosphazide, fozivudine tidoxil, apricitibine (AVX754), GS-7340, KP-1461, or fosalvudine tidoxil (formerly HDP 99.0003),

4) a HIV nucleotide inhibitor of reverse transcriptase, e.g., tenofovir disoproxil fumarate or adefovir dipivoxil,

5) a HIV integrase inhibitor, e.g., curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), elvitegravir, BMS-538158, GSK364735C, BMS-707035, MK-2048, and BA 011, 6) a gp41 inhibitor, e.g., enfuvirtide, sifuvirtide, FB006M, or TRI-1144,

7) a CXCR4 inhibitor, e.g., AMD-070,

8) an entry inhibitor, e.g., SP01A,

9) a gp120 inhibitor, e.g., BMS-488043 or BlockAide/CR,

10) a G6PD and NADH-oxidase inhibitor, e.g., immunitin,

11) a CCR5 inhibitor, e.g., aplaviroc, vicriviroc, maraviroc, PRO-140, INCB15050, PF-232798 (Pfizer), or CCR5mAb004,

12) other drugs for treating HIV, e.g., BAS-100, SPI-452, REP 9, SP-01A, TNX-355, DES6, ODN-93, ODN-112, VGV-1, PA-457 (bevirimat), Ampligen, HRG214, Cytolin, VGX-410, KD-247, AMZ 0026, CYT 99007A-221 HIV, DEBIO-025, BAY 50-4798, MDX010 (ipilimumab), PBS 119, ALG 889, or PA-1050040 (PA-040),

13) an interferon, e.g., pegylated rIFN-alpha 2b, pegylated rIFN-alpha 2a, rIFN-alpha 2b, rIFN-alpha 2a, consensus IFN alpha (infergen), feron, reaferon, intermax alpha, r-IFN-beta, infergen+actimmune, IFN-omega with DUROS, albuferon, locteron, Albuferon, Rebif, Oral interferon alpha, IFNalpha-2b XL, AVI-005, PEG-Infergen, or Pegylated IFN-beta,

14) a ribavirin analog, e.g., rebetol, copegus, viramidine (taribavirin),

15) a NS5b polymerase inhibitor, e.g., NM-283, valopicitabine, R1626, PSI-6130 (R1656), HCV-796, BILB 1941, XTL-2125, MK-0608, NM-107, R7128 (R4048), VCH-759, PF-868554, or GSK625433,

16) A NS3 protease inhibitor, e.g., SCH-503034 (SCH-7), VX-950 (telaprevir), BILN-2065, BMS-605339, or ITMN-191,

17) an alpha-glucosidase 1 inhibitor, e.g., MX-3253 (celgosivir), UT-231B,

18) hepatoprotectants, e.g., IDN-6556, ME 3738, LB-84451, or MitoQ,

19) a non-nucleoside inhibitor of HCV, e.g., benzimidazole derivatives, benzo-1,2,4-thiadiazine derivatives, phenylalanine derivatives, A-831, GS-9190, and A-689; and 20) other drugs for treating HCV, e.g., zadaxin, nitazoxanide (alinea), BIVN-401 (virostat), PYN-17 (altirex), KPE02003002, actilon (CPG-10101), KRN-7000, civacir, GI-5005, ANA-975, XTL-6865, ANA 971, NOV-205, tarvacin, EHC-18, NIM811, DEBIO-025, VGX-410C, EMZ-702, AVI 4065, Bavituximab, Oglufanide, or VX-497 (merimepodib).

In one embodiment the active pharmaceutical agent is selected from darunavir, atazanavir, and elvitegravir.

In one embodiment the active pharmaceutical agent is elvitegravir.

In one embodiment the active pharmaceutical agent is selected from tenofovir disoproxil fumarate, and emtricitabine.

In one embodiment the solid core consists essentially of the active pharmaceutical agent.

In one embodiment, the particles are for use in treatment of a viral disease, such as HIV or HBV.

### Preparation of Particles of the Invention

Particles can be prepared by combining the active pharmaceutical agent, Compound 2, and a suitable organic solvent (e.g. an aprotic organic solvent). For example suitable solvents may comprise heptane, toluene, anisole, chlorobenzene, isopropyl acetate, 2-methyltetrahydrofuran, or methyl tert-butylether, or a mixture thereof. The solvent may also include a suitable anti-solvent. For example, suitable anti-solvents may comprise anisole, chlorobenzene, isopropyl acetate, 2-methyltetrahydrofuran, methyl tert-butylether, or a mixture thereof. The resulting mixture can be maintained at any suitable temperature until the particles form. For example, the mixture can be maintained at a temperature in the range of from about −10° C. to about 40° C. The mixture can be stirred while the particles are forming, but stirring is not always required.

In one embodiment the particles can be prepared by adding a slurry of the active therapeutic agent in heptane to a solution of Compound 2 in toluene; and stirring rapidly at a temperature of 22±5° C. until a uniform off-white slurry containing the particles of the invention forms. The slurry can be stirred using any method that provides the particles of the invention. Typically the slurry is stirred at a speed in the range of from about 500 to about 1200 revolutions per minute. For example, the slurry can be stirred at a speed in the range of from about 700 to about 1100 revolutions per minute, or at a speed in the range of from about 200 to about 500 revolutions per minute.

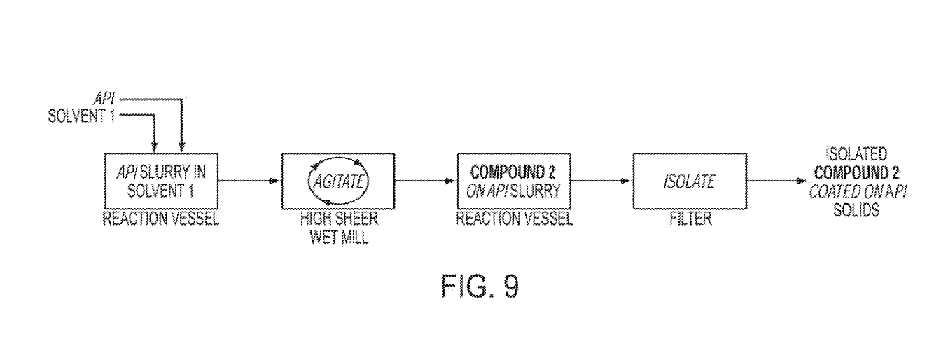

Alternatively, the particles can be prepared by any other suitable method, such as, for example, by high sheer wet milling as illustrated in FIG. 9.

In an embodiment the solid particles have a surface. In one or more embodiments, the solid particles comprise a core having one or more of, a substantially smooth surface, a treated surface, an untreated surface, or pores. Compound 2 may form a coating on the core, for example, but not limited to, on all or a portion of the core or in one or more pores of the core, or a combination thereof. In one embodiment Compound 2 can be adsorbed onto the core.

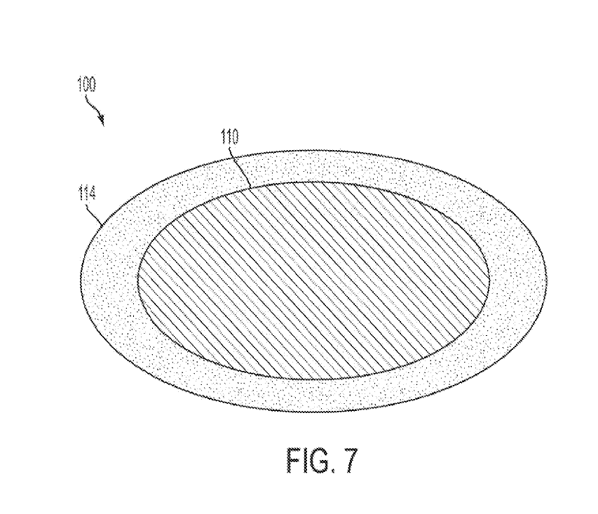

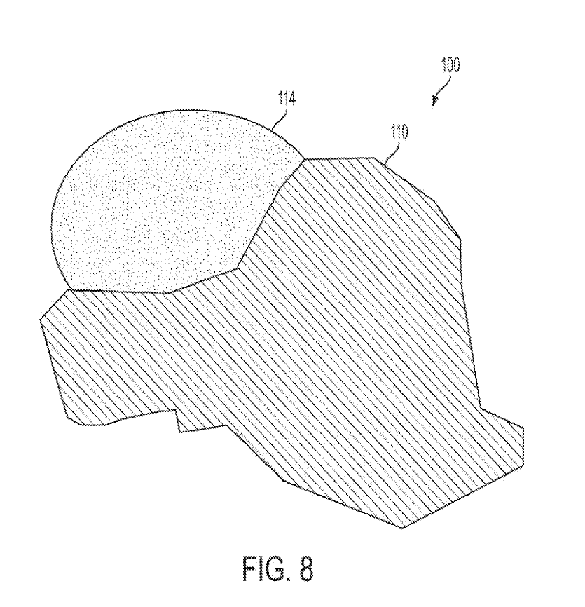

In another embodiment, as shown in FIGS. 7 and 8, a core 110 comprises an active therapeutic agent, and Compound 2 is a coating 114 on, in, or around, the core, or combinations thereof for the composition 100. In another embodiment, the core 110 includes the active therapeutic agent and additional material, and/or the coating 114 includes Compound 2 and additional material to form the composition 100 of the invention. The composition 100 can be used to deliver the compounds in a variety of forms. In an embodiment, the active therapeutic agent forms particles such as, but not limited to, solid particles, for example, at room temperature. The particles can have a variety of shapes such as spherical, partially-spherical and the like. In one embodiment the core 110 has a diameter of about 1 μm to about 1 mm. In another embodiment the core 110 has a diameter of from about 1 μM to about 500 μm.

The shapes are defined in part by an outer surface. The outer surface can have a smooth surface, textured surface, treated surface, untreated surface, coated surface, surface with raised projections, surface with recesses or pores, or combinations thereof.

Compound 2 forms a coating 114 including on, in, or around the core 110 that includes the active therapeutic agent. The coating 114 can be at one or a combination of locations on, in, or around the core 110. The coating 114 of Compound 2, in one or more embodiments, at least partially coats the solid core, and/or fully encapsulates the core, as shown in FIGS. 8 and 7, respectively. The coating 114 is disposed on the core, for instance, as discussed in the Examples herein. In one embodiment the coating 114 has a thickness of about 0.01 μm to about 50 μm. In another one embodiment the coating 114 has a thickness of about 1 μm to about 50 μm. In one embodiment the coating 114 covers at least about 50% of the core 110. In another embodiment the coating 114 covers at least about 75% of the core 110. In another embodiment the coating 114 covers at least about 90% of the core 110. In another embodiment the coating 114 covers at least about 98% of the core 110.

The coating 114 of Compound 2 is disposed on, in, and/or around the core. In an embodiment, the coating 114 of Compound 2 is disposed on the surface or the outer surface of the particle. In another embodiment, the coating 114 of Compound 2 is disposed in the pores of the core. In yet another embodiment, the coating of Compound 2 is disposed in on the surface and one or more pores of the core particles, and optionally encapsulates the core.

One or more embodiments include a method of making a pharmaceutical composition. In an embodiment, the method includes disposing a Compound 2 mixture in to a mixture that includes the active therapeutic agent and forming a combination. In one embodiment of the invention the active therapeutic agent mixture includes the active therapeutic agent and optionally additional material, and/or the Compound 2 mixture includes Compound 2 and optionally additional material. In another embodiment, the method includes, for example, but not limited to, stirring the combination of the Compound 2 mixture and the active therapeutic agent mixture at a predetermined rate, having predetermined amounts of the active therapeutic agent mixture and Compound 2 mixture, predetermined temperature, predetermined amount of time, or combinations thereof. For example, in an embodiment, the method includes stirring the combination of an active therapeutic agent and Compound 2 for at least about 5 hours at any suitable temperature, for example, a temperature of 22±20° C. In an embodiment, the method includes stirring the combination of elvitegravir and Compound 2 in a suitable solvent for 65±20 hours at a temperature of 22±20° C. In an embodiment, the method includes stirring a combination of darunavir and Compound 2 in a suitable solvent for about 15±10 hours at a temperature of about 22±10° C. In an embodiment, the method includes stirring a combination of atazanavir and Compound 2 in a suitable solvent for about 15±10 hours at a temperature of about 22±10° C.

In one or more embodiments, the method further includes filtering, washing, or drying, or combinations thereof. In an embodiment, the method includes filtering solids from the stirred combination of mixtures. In another embodiment, the method includes washing the filtered solids, for example, with a washing medium such as heptane. In another embodiment, the method includes drying the filtered remains, for instance at a temperature higher than room temperature, such as 40° C. In a further embodiment, the drying can our at reduced pressure.

### Additional Materials

In addition to the active therapeutic agent the core 110 can also include other materials, such as pharmaceutically acceptable excipients, solid carrier particles, or polymer materials. In one embodiment the core comprises an active therapeutic agent and a pharmaceutically acceptable excipient.

In one embodiment the core 110 is a solid particle that comprises the active therapeutic agent and a solid carrier particle. For example, the core 110 can comprise a solid carrier particle with the active therapeutic agent coated thereon. For example, suitable solid carrier particles include kaolin, bentonite, hectorite, colloidal magnesium-aluminum silicate, silicon dioxide, magnesium trisilicate, aluminum hydroxide, magnesium hydroxide, magnesium oxide and talc. In one embodiment of the invention, the solid carrier particle can comprise calcium silicate (such as Zeopharm), or magnesium aluminometasilicate (such as Neusilin). In one embodiment the core 110 comprises an active therapeutic agent and a solid carrier particle that comprises silicon dioxide. The active therapeutic agent can be coated in pores and/or on the surface of the solid carrier particle.

Suitable silica derivatives for use in the compositions of the invention and methods for preparing such silica derivatives include those that are described in international patent application publication number WO 03/037379 and the references cited therein. Typically, these silica derivatives comprise a granular hydrophilic fumed silica that has a mean particle diameter of 10 to 120 micron and a BET surface area of 40 to 400 m²/g (determined according to DIN 66 131 with nitrogen). The silica derivatives also typically have a pore volume of about 0.5 to 2.5 mL/g, wherein less than about 5% of the overall pore volume has a pore diameter of less than about 5 nm, the remainder being mesopores and macropores. Additionally, the silica derivatives typically have a pH in the range of about 3.6 to about 8.5 and a tamped density of about 220 to about 700 g/L.

A specific silica material that is particularly useful in the compositions and methods of the invention is AEROPERL® 300 (fumed silica), which is available from Evonik Degussa AG, Dusseldorf, Germany. However, other materials having physical and chemical properties similar to the silica materials described herein can also be used.

In one embodiment of the invention the silica particles have a mean grain diameter of 20-40 micron. In one embodiment of the invention the silica particles have a BET surface area of at least 150 m²/g. In one embodiment of the invention the silica particles have a BET surface area of at least 200 m²/g. In one embodiment of the invention the silica particles have a BET surface area of at least 250 m²/g. In one embodiment of the invention the silica particles have a BET surface area of at least 275 m²/g.

In one embodiment the core 110 is a solid particle that comprises the active therapeutic agent and a high glass transition temperature polymer. In one embodiment, the high glass transition temperature polymer has a glass transition temperature of at least about 100° C. In one embodiment, the high glass transition temperature polymer is selected from the group consisting of HPMC E5, PVP, PVP-VA, HMPC-P and HPMC-AS and mixtures thereof. In one embodiment, the high glass transition temperature polymer is HPMC E5.

In addition to Compound 2, the coating 114 can also include other materials, such as pharmaceutically acceptable excipients or polymer materials.

In one embodiment the coating 114 comprises Compound 2 and a high glass transition temperature polymer. In one embodiment, the high glass transition temperature polymer has a glass transition temperature of at least about 100° C. In one embodiment, the high glass transition temperature polymer is selected from the group consisting of HPMC E5, PVP, PVP-VA, HMPC-P and HPMC-AS and mixtures thereof. In one embodiment, the high glass transition temperature polymer is HPMC E5. In one embodiment the coating 114 consists essentially of Compound 2

### Combination Formulations

In one embodiment, the pharmaceutical compositions comprise, 1) a plurality of solid particles of the invention, and 2) one, two, or three additional therapeutic agents. In another embodiment, the pharmaceutical compositions comprise, 1) a plurality of solid particles of the invention, and 2) two or three additional therapeutic agents. For example, additional therapeutic agents selected from the classes of HIV protease inhibitors, HIV non-nucleoside inhibitors of reverse transcriptase, HIV nucleoside inhibitors of reverse transcriptase, HIV nucleotide inhibitors of reverse transcriptase, and HIV integrase inhibitors. The two or three additional therapeutic agents can be different therapeutic agents selected from the same class of therapeutic agents, or they can be selected from different classes of therapeutic agents.

In another embodiment a pharmaceutical composition comprising a plurality of solid particles that comprise elvitegravir as an active therapeutic agent; tenofovir disoproxil fumarate; emtricitabine; and elvitegravir.

In another embodiment includes a pharmaceutical composition comprising a plurality of solid particles that comprise elvitegravir as an active therapeutic agent; tenofovir alafenamide fumarate GS-7340; and emtricitabine. In another embodiment, the pharmaceutical composition comprises a plurality of particles comprising darunavir as the active therapeutic agent. The composition could include one or more of the following emtricitabine, elvitegravir, tenofovir disoproxil fumarate, tenofovir alafenamide fumarate, tenofovir, darunavir, atazanivir, or rilpiverine in the particle or as separate particles or parts of the composition.

### Combination Methods of Treatment

Co-administration of a composition with one or more other active therapeutic agents generally refers to simultaneous or sequential administration of the composition and one or more other active therapeutic agents, such that therapeutically effective amounts of the composition and one or more other active therapeutic agents are both present in the body of the patient.

Co-administration includes administration of unit dosages of the composition before or after administration of unit dosages of one or more other active therapeutic agents, for example, administration of the composition within seconds, minutes, or hours of the administration of one or more other active therapeutic agents. For example, a unit dose of a composition can be administered first, followed within seconds or minutes by administration of a unit dose of one or more other active therapeutic agents. Alternatively, a unit dose of one or more other therapeutic agents can be administered first, followed by administration of a unit dose of a composition within seconds or minutes. In some cases, it may be desirable to administer a unit dose of a composition first, followed, after a period of hours (e.g., 1 to 12 hours), by administration of a unit dose of one or more other active therapeutic agents. In other cases, it may be desirable to administer a unit dose of one or more other active therapeutic agents first, followed, after a period of hours (e.g., 1 to 12 hours), by administration of a unit dose of a composition.

### Specific Embodiments

Specific embodiments identified herein are for illustration; they do not in any way exclude other embodiments of the invention.

In one embodiment of the invention, Compound 2 is enriched with a stereoisomer of formula 2a:

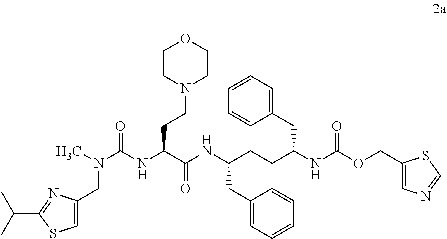 [SMILES: CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

which is (3R,6R,9S)-12-methyl-13-[2-(1-methylethyl)-4-thiazolyl]-9-[2-(4-morpholinyl)ethyl]-8,11-dioxo-3,6-bis(phenylmethyl)-2,7,10,12-tetraazatridecanoic acid, 5-thiazolylmethyl ester. In one embodiment Compound 2 has an enriched concentration of 85±5% of the stereoisomer of formula 2a. In another embodiment Compound 2 has an enriched concentration of 90±5% of the stereoisomer of formula 2a. In another embodiment Compound 2 has an enriched concentration of 95±2% of the stereoisomer of formula 2a. In another embodiment Compound 2 has an enriched concentration of 99±1% of the stereoisomer of formula 2a. In another embodiment Compound 2 contains less than 1% of any stereoisomer other than the stereoisomer of formula 2a. In another embodiment Compound 2 is the pure the stereoisomer of formula (Ia).

The invention will now be illustrated by the following non-limiting Examples.

### EXAMPLES

### Example 1

### Preparation of a Representative Composition of the Invention

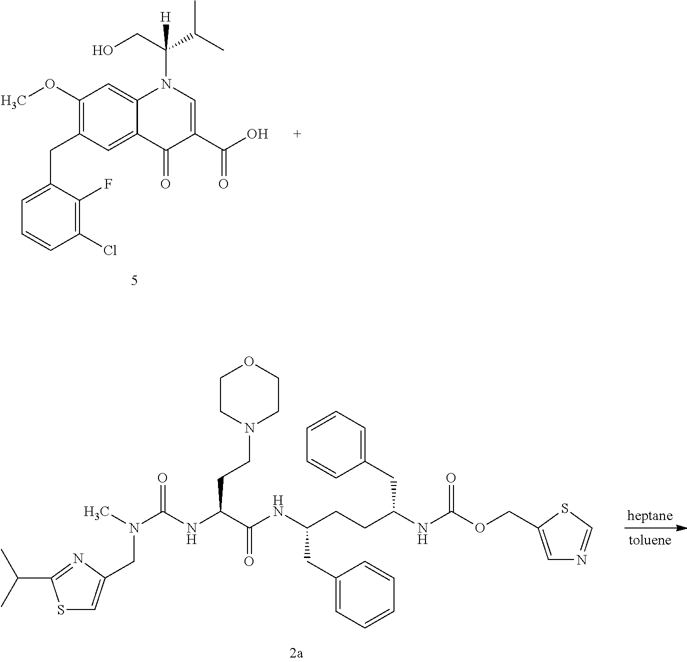 [SMILES: COc1cc2c(cc1Cc1cccc(Cl)c1F)c(=O)c(C(=O)O)cn2[C@H](CO)C(C)C ; CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

To a slurry of elvitegravir 5 (5.0 g) in heptane (150 mL) was added a solution of Compound 2a (5.0 g) in toluene (12.5 mL). The mixture was stirred rapidly at about 22° C. for 66 hours at which time a uniform, off-white slurry was observed. The mixture was filtered and the solid material was washed with heptane (50 mL). The wet cake was thoroughly dried at 40° C. under reduced pressure to afford the particles of the invention as an off-white powder (9.15 g, 92% isolated yield; 1.03:1 (w/w) Compound 5:Compound 2a). HPLC assay (Column: Phenomenex Synergi 4μ MAX RP 80 \acute{Å}; Mobile Phase A: 20 mM ammonium acetate buffer, pH=4.6; Mobile Phase B: acetonitrile): Compound 5: RT=21.9 min (A=7258101); Compound 2a: RT=17.6 min. (A=1904968).

### Example 2

### Physicochemical Evaluation

### Thermal Analysis by DSC and TGA

### Procedure DSC:

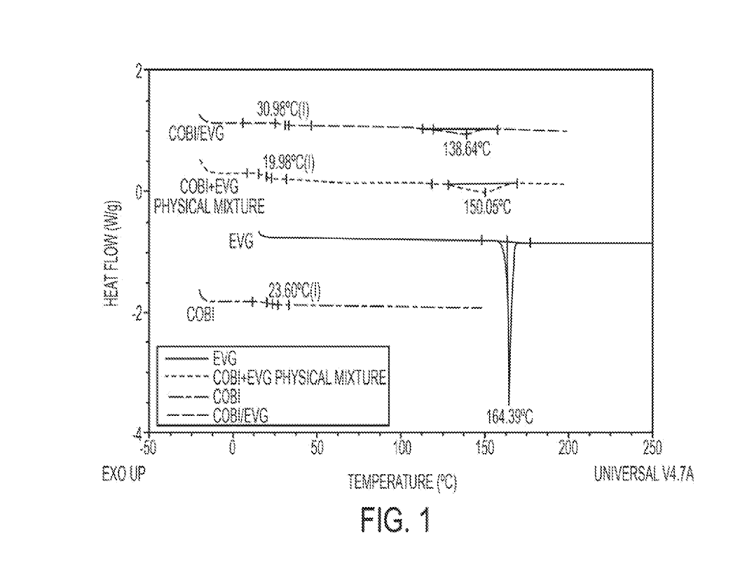

The thermal events (glass transition temperature and melting point) were determined by differential scanning calorimetry (TA Instruments, New Castle, Del., USA, Model 1000) in which 5 to 10 mg of solid a) Compound 2a, b) Compound 5, c) Compound 5 and Compound 2a physical mixture or d) the product of Example 1 were placed in a hermetically sealed aluminum pan with a pinhole and heated at rate of 10° C./min under dried nitrogen purge. Results are shown in FIG. 1.

### Procedure TGA:

Thermal gravimetric analysis (TGA) measures the weight loss upon heating and was conducted with the product of Example 1 in an open aluminum pan; the sample was heated at a rate of 10° C./min (TA Instruments, New Castle, Del., USA, Model 500).

### Results

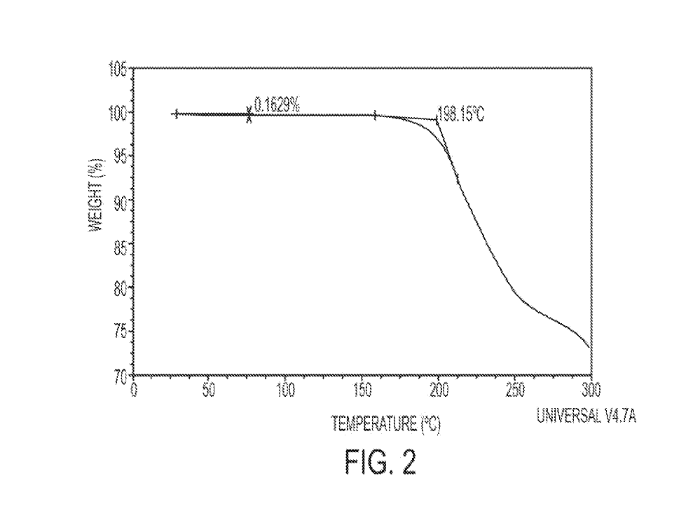

The product of Example 1 has two thermal events in the temperature range of 0-200° C. (see FIG. 2). The first event is characteristic of a glass transition temperature that is typically observed with 20-35° C. (depending on initial moisture content). The second event is a broad endotherm that is associated with melting of Compound 5. The melting point was observed around 138° C. Melting point for Compound 5 (γ form) alone was noted at 164° C. Depression of the Compound 5 melting point was also observed when a physical mixture of Compound 5 and Compound 2a were analyzed using DSC; confirming XRPD results that there was no Compound 5 form change but rather a physical interaction between Compound 2a and Compound 5 during the experiment.

TGA results confirm that the product of Example 1 is mostly anhydrous (0.16% moisture) with degradation temperature of 198° C.; which is consistent with degradation temperature for Compound 2a.

### Hygroscopicity

### Procedure:

Hygroscopicity of the product of Example 1 was studied by dynamic vapor sorption (DVS) using an automated vapor sorption balance (DVS-1.0 Advantage; Surface Measurement Systems, Ltd. NA, Allentown, Pa.). The first step of the DVS experiment involved equilibrating Compound 2a at 0% RH until a constant weight was achieved. The time and weight of the sample after equilibration at 0% RH were taken as the starting experimental values for the dry mass.

The hygroscopicity of the product of Example 1 as a function of relative humidity (RH) over the range of 0 to 90% in 10% increments was monitored. For desorption the relative humidity was deceased in a similar manner to accomplish a full sorption/desorption cycle. For comparison purposed hygroscopicity of Compound 2a and Compound 5 were measured in similar manner.

### Results

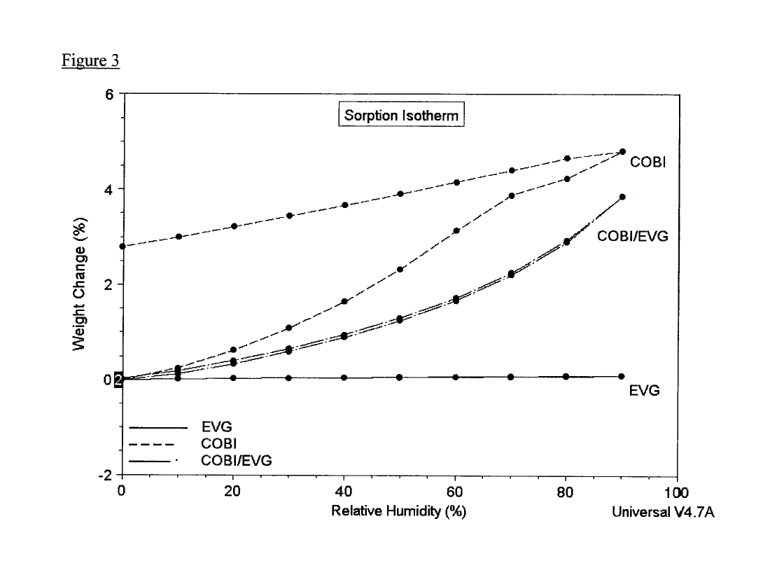

The hygroscopicity of the product of Example 1 is significantly higher than Compound 5 alone but is less hygroscopic than Compound 2a. Surprisingly, the product of Example 1 isotherm does not exhibit hysteresis which means that water sorption and desorption on the product of Example 1 is a reversible process, unlike for Compound 2a. See FIG. 3.

### Morphology

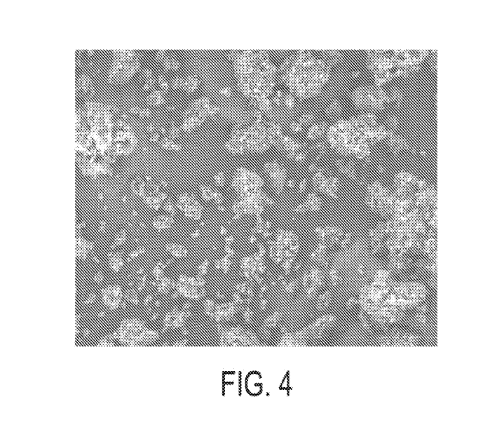

Morphology of the product of Example 1 was observed using SEM. A picture with a 500× magnification is shown in FIG. 4.

### Powder X-Ray Diffraction

### Procedure:

The powder X-ray diffraction of the product of Example 1 was determined at generator tension of 40 KV and current of 10 mA using X'Pert Pro PANalytical. The samples were pressed onto X-ray sample holders and scanned at a step size of 0.008 for 10 minutes. The data were collected from 2Theta range of 2˜40°.

### Results

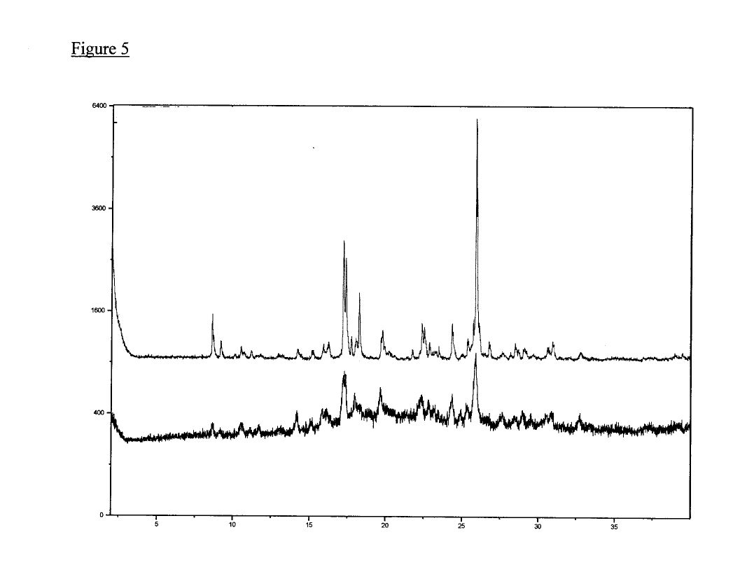

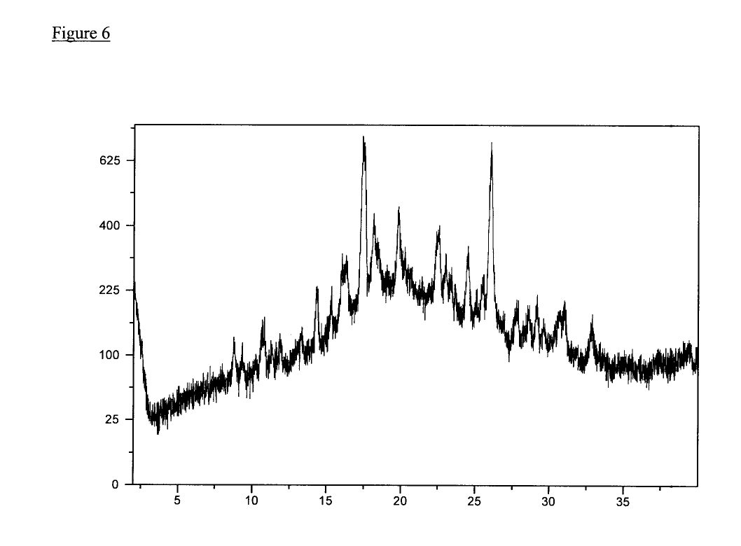

The product of Example 1 has an XRPD pattern consistent with γ form of Compound 5 and contains amorphous material (consistent with amorphous form of Compound 2a). See FIGS. 5 and 6.

### Example 3

### Preparation of a Representative Composition of the Invention

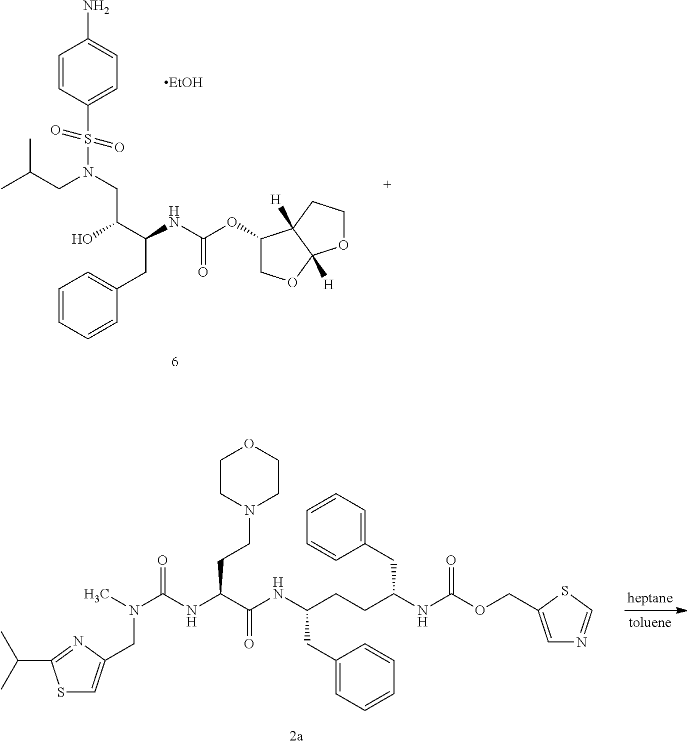 [SMILES: "·EtOH" ; CC(C)CN(C[C@@H](O)[C@H](Cc1ccccc1)NC(=O)O[C@H]1CO[C@H]2OCC[C@H]21)S(=O)(=O)c1ccc(N)cc1 ; CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

To a slurry of darunavir 6 (3.0 g) in heptane (60 mL) was added a 34.9 wt % stock solution of Compound 2a in toluene (1.57 g). The mixture was stirred rapidly at about 22° C. for 15 hours at which time a uniform, bright-white slurry was observed.

The mixture was filtered and the solid material was washed with heptane (2×10 mL) The wet cake was thoroughly dried at 40° C. under reduced pressure to afford particles of the invention as a bright-white powder (3.32 g, 94% isolated yield; 5.5:1 (w/w) Compound 6:Compound 2a). HPLC assay (Column: Phenomenex Synergi 4μ MAX RP 80 \acute{Å}; Mobile Phase A: 20 mM ammonium acetate buffer, pH=4.6; Mobile Phase B: acetonitrile): Compound 2a: RT=17.0 min. (A=3664581); Compound 6: RT=16.3 min (A=8872291)

### Example 4

### Preparation of a Representative Composition of the Invention

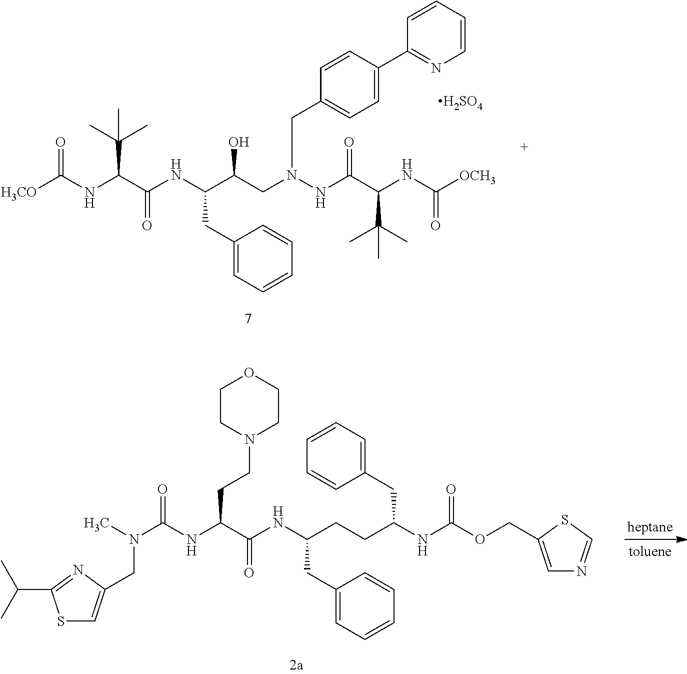

To a slurry of atazanavir 7 (1.0 g) in heptane (20 mL) was added a solution of Compound 2a (0.5 g) in toluene (1 mL). The mixture was stirred rapidly at about 22° C. for 15 hours at which time a uniform, off-white slurry was observed. The mixture was filtered and the solid material was washed with heptane (2×9 mL). The wet cake was thoroughly dried at 40° C. under reduced pressure to give particles of the invention as an off-white powder (1.38 g, 91% isolated yield; 2.2:1 (w/w) atazanavir:cobicistat). HPLC assay (Column: Phenomenex Synergi 4μ MAX RP 80 \acute{Å}; Mobile Phase A: 20 mM ammonium acetate buffer, pH=4.6; Mobile Phase B: acetonitrile): Compound 2a: RT=17.6 min. (A=1026219); Compound 7: RT=19.2 min (A=3118235)

### Example 5

### Preparation of a Representative Composition of the Invention

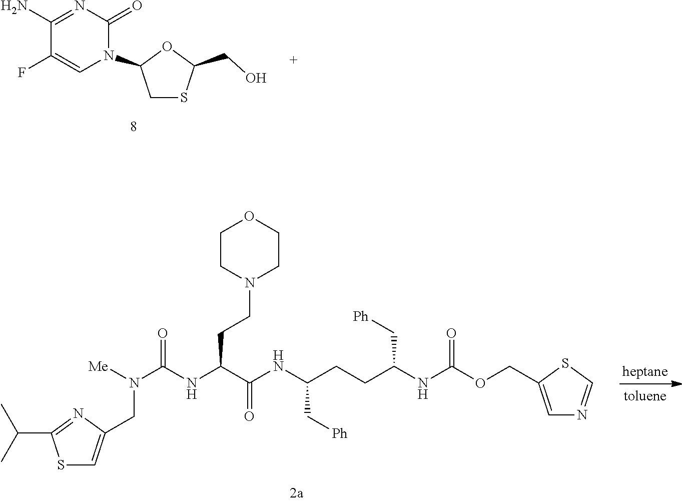 [SMILES: Nc1nc(=O)n([C@@H]2CS[C@H](CO)O2)cc1F ; CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

To a slurry of emtricitabine 8 (2.6 g) in heptane (52 mL) was added a 35 wt % stock solution of 2a in toluene (5.61 g). The mixture was stirred rapidly at 22° C. for 72 hours at which time a uniform off-white slurry was observed. The mixture was then filtered and the solid material was washed with heptane (25 mL). The wet cake was then thoroughly dried at 40° C. under reduced pressure to give the particles of the invention as an off-white powder (3.86 g, 85% isolated yield; 1.3:1 (w/w) Compound 8:Compound 2a). HPLC assay (Column: Phenomenex Synergi 4μ MAX RP 80 \acute{Å}; Mobile Phase A: 20 mM ammonium acetate buffer, pH=4.6; Mobile Phase B: acetonitrile): Compound 2a: RT=16.9 min. (A=10038094); Compound 8: RT=2.8 min (A=20423584).

### Example 6

### Preparation of a Representative Composition of the Invention

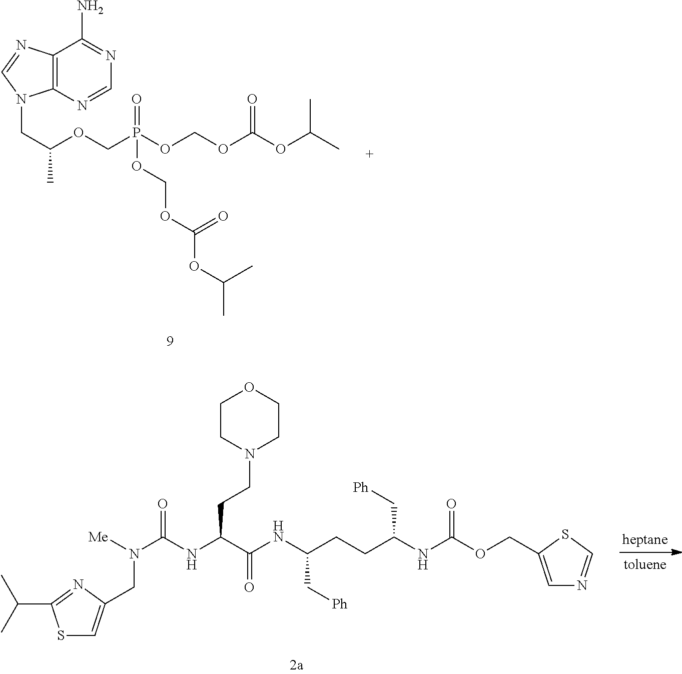 [SMILES: CC(C)OC(=O)OCOP(=O)(CO[C@H](C)Cn1cnc2c(N)ncnc21)OCOC(=O)OC(C)C ; CC(C)c1nc(CN(C)C(=O)N[C@@H](CCN2CCOCC2)C(=O)N[C@H](CC[C@H](Cc2ccccc2)NC(=O)OCc2cncs2)Cc2ccccc2)cs1]

To a slurry of tenofovir disoproxil fumerate 9 (3.0 g) in heptane (60 mL) was added a 35 wt % stock solution of 2a in toluene (4.33 g). The mixture was stirred rapidly at 22° C. for 72 hours at which time a uniform off-white slurry was observed. The mixture was then filtered and the solid material was washed with heptane (25 mL). The wet cake was then thoroughly dried at 40° C. under reduced pressure to give the particles of the invention as an off-white powder (4.13 g, 92% isolated yield; 2:1 (w/w) Compound 9:Compound 2a). HPLC assay (Column: Phenomenex Synergi 4μ MAX RP 80 \acute{Å}; Mobile Phase A: 20 mM ammonium acetate buffer, pH=4.6; Mobile Phase B: acetonitrile): Compound 2a: RT=16.9 min. (A=7045702); Compound 9: RT=11.9 min (A=9074961).

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A particle comprising: a) a solid core that comprises an active pharmaceutical agent, and b) a coating of Compound 2: 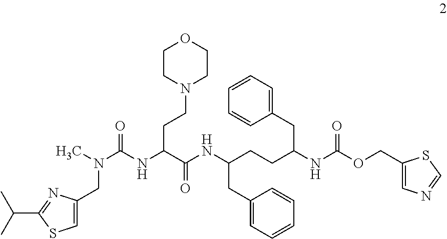 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]
or a pharmaceutically acceptable salt of thereof on the core.

2. The particle of claim 1 wherein the active pharmaceutical agent is an agent that is metabolized by cytochrome P450 monooxygenase when administered to an animal.

3. The particle of claim 1 wherein the active pharmaceutical agent is an agent that is not metabolized by cytochrome P450 monooxygenase when administered to an animal.

4. The particle of claim 1 wherein the active pharmaceutical agent is selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, interferons, ribavirin analogs, NS3 protease inhibitors, alpha-glucosidase 1 inhibitors, hepatoprotectants, non-nucleoside inhibitors of HCV, and other drugs for treating HCV, and combinations thereof.

5. The particle of claim 1 wherein the active pharmaceutical agent is a protease inhibitor.

6. The particle of claim 1 wherein the active pharmaceutical agent is an integrase inhibitor.

7. The particle of claim 1 wherein the active pharmaceutical agent is:
1) amprenavir, atazanavir, fosamprenavir, indinavir, lopinavir, ritonavir, nelfinavir, saquinavir, tipranavir, brecanavir, darunavir, TMC-126, TMC-114, mozenavir (DMP-450), JE-2147 (AG1776), L-756423, RO0334649, KNI-272, DPC-681, DPC-684, GW640385X, DG17, GS-8374, PPL-100, DG35, or AG 1859,
2) a HIV non-nucleoside inhibitor of reverse transcriptase, e.g., capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirene), efavirenz, BILR 355 BS, VRX 840773, UK-453061, or RDEA806,
3) a HIV nucleoside inhibitor of reverse transcriptase, e.g., zidovudine, emtricitabine, didanosine, stavudine, zalcitabine, lamivudine, abacavir, amdoxovir, elvucitabine, alovudine, MIV-210, racivir (±-FTC), D-d4FC, emtricitabine, phosphazide, fozivudine tidoxil, apricitibine (AVX754), GS-7340, KP-1461, or fosalvudine tidoxil (formerly HDP 99.0003),
4) a HIV nucleotide inhibitor of reverse transcriptase, e.g., tenofovir disoproxil fumarate or adefovir dipivoxil,
5) a HIV integrase inhibitor, e.g., curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), elvitegravir, BMS-538158, GSK364735C, BMS-707035, MK-2048, or BA 011,
6) a gp41 inhibitor, e.g., enfuvirtide, sifuvirtide, FB006M, or TRI-1144,
7) a CXCR4 inhibitor, e.g., AMD-070,
8) an entry inhibitor, e.g., SP01A,
9) a gp120 inhibitor, e.g., BMS-488043 or BlockAide/CR,
10) a G6PD and NADH-oxidase inhibitor, e.g., immunitin,
11) a CCR5 inhibitor, e.g., aplaviroc, vicriviroc, maraviroc, PRO-140, INCB15050, PF-232798, or CCR5mAb004,
12) other drugs for treating HIV, e.g., BAS-100, SPI-452, REP 9, SP-01A, TNX-355, DES6, ODN-93, ODN-112, VGV-1, PA-457 (bevirimat), Ampligen, HRG214, Cytolin, VGX-410, KD-247, AMZ 0026, CYT 99007A-221 HIV, DEBIO-025, BAY 50-4798, MDX010 (ipilimumab), PBS 119, ALG 889, or PA-1050040 (PA-040),
13) an interferon, e.g., pegylated rIFN-alpha 2b, pegylated rIFN-alpha 2a, rIFN-alpha 2b, rIFN-alpha 2a, consensus IFN alpha (infergen), feron, reaferon, intermax alpha, r-IFN-beta, infergen+actimmune, IFN-omega with DUROS, albuferon, locteron, Albuferon, Rebif, Oral interferon alpha, IFNalpha-2b XL, AVI-005, PEG-Infergen, or Pegylated IFN-beta,
14) a ribavirin analog, e.g., rebetol, copegus, viramidine (taribavirin),
15) a NS5b polymerase inhibitor, e.g., NM-283, valopicitabine, R1626, PSI-6130 (R1656), HCV-796, BILB 1941, XTL-2125, MK-0608, NM-107, R7128 (R4048), VCH-759, PF-868554, or GSK625433,
16) A NS3 protease inhibitor, e.g., SCH-503034 (SCH-7), VX-950 (telaprevir), BILN-2065, BMS-605339, or ITMN-191,
17) an alpha-glucosidase 1 inhibitor, e.g., MX-3253 (celgosivir), UT-231B,
18) hepatoprotectants, e.g., IDN-6556, ME 3738, LB-84451, and MitoQ, or
19) a non-nucleoside inhibitor of HCV, e.g., benzimidazole derivatives, benzo-1,2,4-thiadiazine derivatives, phenylalanine derivatives, A-831, GS-9190, and A-689; and 20) other drugs for treating HCV, e.g., zadaxin, nitazoxanide (alinea), BIVN-401 (virostat), PYN-17 (altirex), KPE02003002, actilon (CPG-10101), KRN-7000, civacir, GI-5005, ANA-975, XTL-6865, ANA 971, NOV-205, tarvacin, EHC-18, NIM811, DEBIO-025, VGX-410C, EMZ-702, AVI 4065, Bavituximab, Oglufanide, or VX-497 (merimepodib).

8. The particle of claim 1 wherein the active pharmaceutical agent is darunavir, atazanavir, or elvitegravir.

9. The particle of claim 1 wherein the active pharmaceutical agent is darunavir.

10. The particle of claim 1 wherein the active pharmaceutical agent is elvitegravir.

11. The particle of claim 1 wherein the active pharmaceutical agent is tenofovir disoproxil fumarate or emtricitabine.

12. The particle of claim 1 wherein the solid core consists essentially of the active pharmaceutical agent.

13. The particle of claim 1 wherein the solid core has a diameter of about 1 μm to about 1 mm.

14. The particle of claim 1 wherein the solid core has a diameter of from about 1 μm to about 500 μm.

15. The particle of claim 1 wherein the coating has a thickness of about 0.01 μm to about 50 μm.

16. The particle of claim 1 wherein the coating has a thickness of about 1 μm to about 50 μm.

17. The particle of claim 1 wherein the coating covers at least about 75% of the core.

18. The particle of claim 1 wherein the coating covers at least about 90% of the core.

19. The particle of claim 1 wherein the core comprises the active therapeutic agent and a pharmaceutically acceptable excipient.

20. The particle of claim 1, wherein the core comprises the active therapeutic agent and a solid carrier particle.

21. The particle of claim 1, wherein the core comprises the active therapeutic agent and a solid carrier particle that comprises kaolin, bentonite, hectorite, colloidal magnesium-aluminum silicate, silicon dioxide, magnesium trisilicate, aluminum hydroxide, magnesium hydroxide, magnesium oxide or talc.

22. The particle of claim 1 wherein the core comprises the active therapeutic agent and a solid carrier particle that comprises silicon dioxide.

23. A composition comprising the particle of claim 1 and a pharmaceutically acceptable excipient.

24. A method for treating an HIV infection comprising administering to a patient in need thereof a therapeutically effective amount of a composition as described in claim 23.

25. A pharmaceutical composition comprising a plurality of solid particles as described in claim 10; tenofovir disoproxil fumarate; and emtricitabine.

26. A pharmaceutical composition comprising a plurality of solid particles as described in claim 10; GS-7340; and emtricitabine.

27. A composition as described in claim 23 for use in medical therapy.

28. The use of a composition as described in claim 23 for the prophylactic or therapeutic treatment of an HIV infection.

29. A composition as described in claim 23 for use in the preparation of a medicament for treating HIV infection in a mammal.

30. A method comprising: a) combining an active pharmaceutical agent and Compound 2: 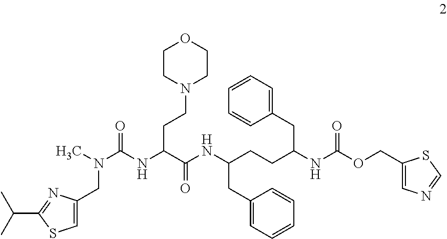 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]
in a suitable solvent to provide a mixture; and b) maintaining the mixture under conditions suitable to provide one or more solid particles comprising a solid core that comprises the active pharmaceutical agent and that has a coating of Compound 2.

31. A method for preparing a pharmaceutical composition comprising: combining a plurality of solid particles as described in claim 10, tenofovir disoproxil fumarate, and emtricitabine to provide the pharmaceutical composition.

32. A pharmaceutical composition comprising:
a core comprising 6-(3-chloro-2-fluorobenzyl)-1-[(2S)-1-hydroxy-3-methylbutan-2-yl]-7-methoxy-4-oxo-1,4-dihydroquinoline-3-carboxylic acid or a salt thereof; and
the core having a coating including Compound 2: 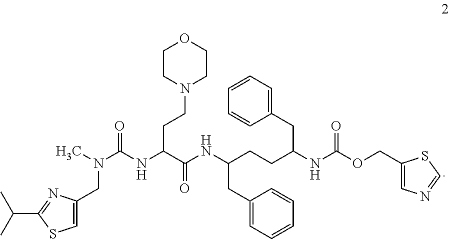 [SMILES: CC(C)c1nc(CN(C)C(=O)NC(CCN2CCOCC2)C(=O)NC(CCC(Cc2ccccc2)NC(=O)OCc2cncs2)Cc2ccccc2)cs1]
